(19) United States
(12) Patent Application Publication
(10) Pub. No.: US 2007/0172515 A1
(43) Pub. Date: Jul. 26, 2007

(54) Film bandage for mucosal administration of actives

(75) Inventor: Richard C. Fuisz, McLean, VA (US)

Correspondence Address: HOFFMANN & BARON, LLP, 6900 JERICHO TURNPIKE, SYOSSET, NY, 11791, US

(73) Assignee: MonoSolRx, LLC

(21) Appl. No.: 11/655,372

(22) Filed: Jan. 19, 2007

Related U.S. Application Data
(60) Provisional application No. 60/760,563, filed on Jan. 20, 2006.

(51) Int. Cl. A61K 38/28; A61K 9/70

(52) U.S. Cl. 424/443; 514/3

(57) ABSTRACT

The present invention relates to multi-component delivery systems that adhere to mucosal tissue. In particular, the delivery systems include a first delivery vehicle and a second delivery vehicle, which is in association with the first delivery vehicle. The first delivery vehicle may be one or more mucoadhesive films, which may adhere to the mucosal tissue. The second delivery vehicle may contain at least one active substance, such as a pharmaceutical active, for delivery via the mucosal tissue.

### CROSS-REFERENCE TO RELATED APPLICATIONS

The present application claims the benefit of U.S. Provisional Application No. 60/760,563, filed Jan. 20, 2006, the contents of which are incorporated herein by reference.

### FIELD OF THE INVENTION

The present invention relates to multi-component delivery systems that adhere to mucosal tissue. More specifically, the delivery systems include a first delivery vehicle, which may be one or more mucoadhesive films, and a second delivery vehicle, which may be in association with the first delivery vehicle and may include an active.

### BACKGROUND OF THE RELATED TECHNOLOGY

It is often desirable to administer active components via the mucosal tissue in the oral cavity, as opposed to administration through the gastrointestinal tract (GI tract). In particular, a number of drugs, such as insulin, exhibit poor absorption or degrade in the gastrointestinal system. Conventional drug delivery routes, therefore, are not as useful for these types of drugs. Administration via the mucosal tissue is more useful for such drugs because it permits the drug to absorb directly into the bloodstream through the tissue, and avoids the acidic and enzymatic processes of the gut.

Drug delivery systems that deliver actives via mucosal tissue, such as buccally, are known. Such delivery systems, however, often do not completely dissolve, which requires removal of the remaining material from the buccal cavity once the active has been delivered. Additionally, some delivery systems exhibit poor adherence to the mucosal tissue, which makes it difficult for substantial or complete delivery of the active contained therein.

Although it may be desirable to use conventional drug delivery formats in the buccal cavity, such as, for example, oral tablets, these formats typically will not adhere to mucosal surfaces. Further, such delivery formats often dissolve too rapidly, or in an uncontrolled manner, for effective delivery of an active over a period of time, which is desired for mucosal administration.

There is a need, therefore, for delivery systems that adhere to mucosal tissue, particularly for buccal administration of active components, and which can provide controlled release of the actives over time. Such delivery systems may be adapted to further include conventional delivery formats, such as tablets or capsules.

### SUMMARY OF THE INVENTION

In some embodiments, there is provided a mucoadhesive film that is substantially free of active, the film being adapted to accommodate inclusion of a delivery vehicle, such as a tablet, capsule, another film, powder, gel, liquid or any combination thereof. Desirably, the mucoadhesive film physically delivers the second delivery vehicle, which contains at least one active, to the mucosal tissue.

In accordance with some embodiments of the present invention, there is provided a multi-vehicle delivery system including: (a) a first delivery vehicle including at least one mucoadhesive film; and (b) a second delivery vehicle containing at least one active component, wherein the second delivery vehicle is in association with the first delivery vehicle.

In accordance with some other embodiments, there is provided a multi-vehicle delivery system, which includes: (a) a first delivery vehicle including at least one mucoadhesive film; and (b) a second delivery vehicle containing at least one active component, wherein the second delivery vehicle is adjacent to the first delivery vehicle.

Other embodiments of the present invention provide a multi-vehicle delivery system including: (a) a first delivery vehicle including at least one mucoadhesive film, the first delivery vehicle having a cavity defined therein for accommodating a second delivery vehicle; and (b) a second delivery vehicle positioned within the cavity, the second delivery vehicle containing at least one active component.

Some other embodiments described herein provide a consumable product, which includes:

(a) an outer container having one or more compartments; and

(b) a multi-vehicle delivery system housed in the one or more compartments, wherein the delivery system includes:
(i) a first delivery vehicle including at least one mucoadhesive film, the first delivery vehicle having a cavity defined therein for accommodating a second delivery vehicle; and
(ii) a second delivery vehicle positioned within the cavity, the second delivery vehicle containing at least one active component.

In another aspect of the present invention, there is provided a method of making a multi-vehicle delivery system, which includes the steps of:

(a) providing a first delivery vehicle including a mucoadhesive film;

(b) forming a cavity in the mucoadhesive film; and

(c) positioning a second delivery vehicle within the cavity, wherein the second delivery vehicle contains at least one active component.

### BRIEF DESCRIPTION OF THE DRAWINGS

FIG. 1 is a cross-sectional view of a delivery system in accordance with an embodiment of the present invention;

FIG. 2 is a cross-sectional view of a delivery system in accordance with another embodiment of the present invention;

FIG. 3 is a cross-sectional view of a delivery system in accordance with another embodiment of the present invention;

FIG. 4 is a top plan view of a delivery system in accordance with another embodiment of the present invention;

FIG. 4a is a cross-sectional view taken along line 4a-4a of FIG. 4; and

FIG. 5 is a side elevation view of a delivery system in accordance with another embodiment of the present invention.

### DETAILED DESCRIPTION OF THE INVENTION

The present invention relates to multi-component delivery systems that adhere to mucosal tissue. The delivery systems may be used for administration of actives, such as, for example, buccal administration of drugs. In some embodiments, the delivery system may include a first delivery vehicle, which may be one or more mucoadhesive films, and which may be substantially free of active. The mucoadhesive films may be adapted to accommodate inclusion of another delivery vehicle, such as, for example, a tablet. In particular, the delivery system also may include a second delivery vehicle. In some embodiments, the second delivery vehicle may be different from the first delivery vehicle. The second delivery vehicle may include at least one active component. Desirably, the second delivery vehicle may be associated with the first delivery vehicle in a variety of manners. For instance, the second delivery vehicle may be surrounded by the first delivery vehicle, located within a cavity in the first delivery vehicle or positioned adjacent to the first delivery vehicle, among others.

### Delivery Systems

As mentioned above, the delivery systems include a first delivery vehicle and a second delivery vehicle, which may be in association with the first delivery vehicle. The first delivery vehicle may be any mucosal delivery system, such as, one or more mucoadhesive films or a mucoadhesive system that is sponge-like, which, in some embodiments, may be substantially free of active. As used herein, the term “mucoadhesive” refers to materials that adhere to mucosal tissue surfaces. Examples of mucosal tissue surfaces include buccal, vaginal and rectal, among others. The first delivery vehicle, therefore, may be one or more films that adhere to mucosal tissue surfaces.

The second delivery vehicle may be any oral delivery vehicle used to administer actives. In some embodiments, the second delivery vehicle may be, but is not limited to, a tablet, capsule, another film, powder, gel, liquid or any combination thereof. In some embodiments, the second delivery vehicle may be different from the first delivery vehicle, i.e., a delivery vehicle other than another film. The second delivery vehicle may contain at least one active component. Upon administration, the mucosal delivery system, e.g., film, of the first delivery vehicle may adhere to the mucosal tissue, thereby allowing the active contained in the second delivery vehicle to penetrate the mucosal tissue and enter the bloodstream. The mucosal delivery system, e.g., film, may dissolve and/or disintegrate over time in the presence of moisture at the administration site in the body. Once mucosal delivery of the active is complete, the mucosal delivery system, e.g., film, may have substantially or completely dissolved and/or disintegrated.

In some embodiments, the first delivery vehicle may be substantially free of actives. Alternatively, in some embodiments, the first delivery vehicle also may include an active component, which may be the same or different from the active component contained in the second delivery vehicle.

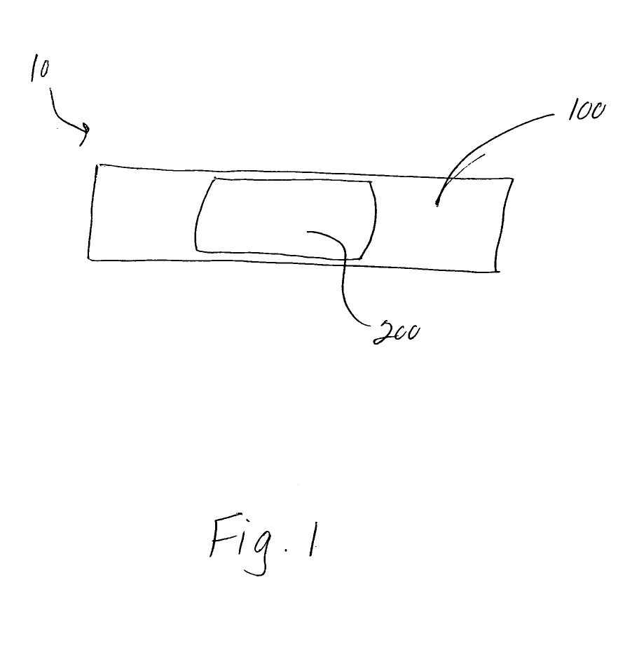

In some embodiments described herein, the second delivery vehicle may be positioned within the first delivery vehicle. More specifically, the first delivery vehicle may be formed around the second delivery vehicle to partially or completely surround the second delivery vehicle. As shown in FIG. 1, for instance, the delivery system 10 includes a film 100 that completely surrounds a second delivery vehicle 200. In some embodiments, for example, the second delivery vehicle 200 may be a tablet, which is fully encompassed by the film 100.

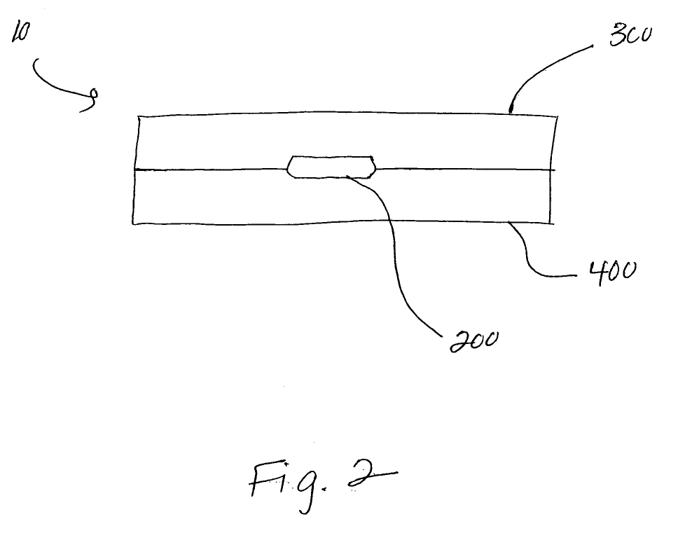

In some other embodiments, the first delivery vehicle may include multiple film layers. For instance, two films may be positioned in at least partial face-to-face engagement with each another. One or both of the films may be mucoadhesive. The second delivery vehicle may be positioned between the films. For example, as shown in FIG. 2, the delivery vehicle 10 may include a first film layer 300 and a second film layer 400. Film layer 300 and film layer 400 may be positioned in partial face-to-face engagement with one another. The second delivery vehicle 200 may be located between film layer 300 and film layer 400. Film layer 300 and film layer 400 may be sealed or fused to each other along the face-to-face engagement, thereby fully surrounding the second delivery vehicle. In particular, the film layers may be heat-sealable.

In some other embodiments, the first delivery vehicle may include a mucoadhesive film having a cavity region therein. The cavity may be a closed cavity defined within the film or an open cavity, which may have at least one open exterior surface. For instance, the open cavity may be an indentation in the film surface. The size and shape of the cavity may vary depending on the size and shape of the second delivery vehicle selected to be located therein. The second delivery vehicle may be positioned within the cavity in the film for administration to the mucosal tissue.

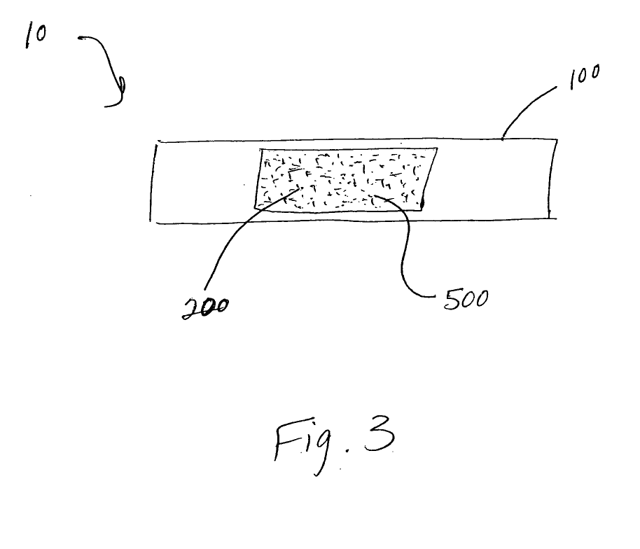

In some embodiments, for instance, as depicted in FIG. 3, the delivery system 10 may include a film 100 and a cavity 500 defined therein. Cavity 500 may be a closed cavity region, as shown in FIG. 3. The second delivery vehicle may be positioned within the closed cavity 500. For example, as shown in FIG. 3, the second delivery vehicle 200 may be a powdered form of an active component, which is located within the closed cavity 500 of the film 100.

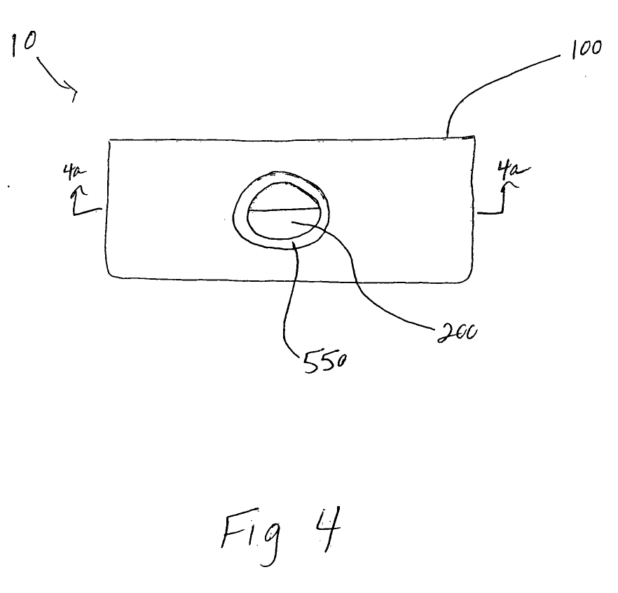

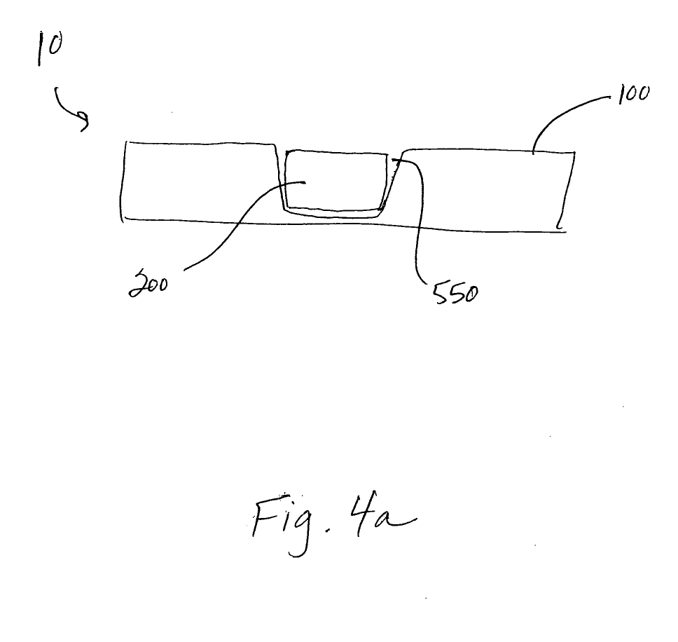

Alternatively, as shown in FIG. 4, the film 100 may include an open cavity 550 defined therein. In such embodiments, the open cavity 550 may be an indentation in the surface of the film 100. As such, the cavity has one open exterior surface. The second delivery vehicle may be located within the open cavity region. Some embodiments further may include a material that covers the open cavity. Any material that is edible and water-soluble may be employed. For example, any of the water-soluble polymers described below may be suitable for use in forming the cover.

For example, as depicted in FIGS. 4 and 4a, the second delivery vehicle 200 is positioned within an open cavity 550 in the mucoadhesive film 100. The open cavity 550 shown in FIG. 4 may have a size and shape suitable for any conventional oral tablet. In such embodiments, it may be desirable to administer the delivery system with the exposed surface of the open cavity placed against the mucosal tissue. Such administration may place the second delivery vehicle into direct contact with the mucosal tissue, thereby permitting the active contained therein to immediately commence penetration of the tissue. Meanwhile, the film may adhere to the tissue and maintain the second delivery vehicle in contact therewith as the active is delivered.

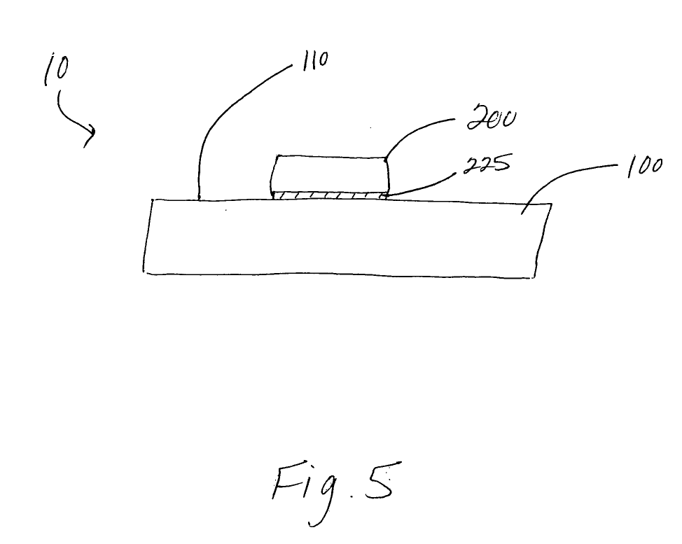

Some other embodiments described herein provide delivery systems in which the second delivery vehicle is adjacent to the first delivery vehicle. In such embodiments, for example, the second delivery vehicle may be adhered to the surface of the first delivery vehicle. As shown in FIG. 5, for example, the delivery system 10 may include a first delivery vehicle, which may be a mucoadhesive film 100. The mucoadhesive film may have opposing top and bottom surfaces. A second delivery vehicle may be positioned adjacent to either surface of the film. As shown in FIG. 5, for example, a second delivery vehicle 200 may be positioned adjacent to the top surface 110 of the film 100. Further, the second delivery vehicle 200 may be adhered to the top surface 110 of the film 100 at the point of contact 225. An adhesive may be used to attach the second delivery vehicle to the film, which may be any of those known in the art. If an adhesive is used, it will desirably be a food-grade adhesive that is ingestible and does not alter the properties of the active.

In some other embodiments, the first delivery vehicle may include a film and a sponge-like material. Any conventional sponge materials may be employed. One or both of the film and the sponge material may be mucoadhesive. The sponge material may be positioned in association with the film. For instance, the film and the sponge material may be separate layers that are positioned adjacent to one another, and may be in at least partial face-to-face engagement with each other. In some embodiments, the sponge material may be affixed or adhered to the front or the back of the film. For example, the sponge material may form a backing for the film. Any conventional material may be used to adhere the sponge material to the film. Additionally, the second delivery vehicle may be positioned in association with the sponge material in any of the formats discussed above regarding films.

The sponge material may be incorporated into the delivery system to hold a reservoir of any component that effects absorption of actives, such as by increasing or prolonging absorption. Accordingly, in some embodiments, the sponge material may include a component that enhances absorption of the active component contained in the second delivery vehicle. For example, the sponge material may include a pH adjuster or a component that creates effervescence upon administration at the desired site in the body.

As mentioned above, the delivery system may be configured to effervesce when positioned at the desired administration site in the body, such as within the oral cavity. Effervescence may provide increased absorption of the active component(s) contained in the delivery system. In particular, effervescence may be provided by the presence of an edible acid in one of the delivery vehicles and a base in the other delivery vehicle. For instance, an edible acid may be included in a mucoadhesive film to activate a base present in an active component contained in the second delivery vehicle. In some other embodiments, an edible acid may be included in a sponge-like material, which is affixed to a mucoadhesive film, to activate a base present in an active component contained in the second delivery vehicle. When the delivery vehicle is positioned at the desired administration site, such as within the oral cavity, moisture at the site may cause the delivery vehicle to dissolve and the acid and base will react to produce effervescence.

In some other embodiments, the entire delivery system or one of the delivery vehicles may be dipped into an edible acid and/or base to activate an acid or base present in another portion of the delivery system. For example, a sponge material may be dipped in an edible acid and then affixed to the back of a mucoadhesive film. A second delivery vehicle, such as a tablet that includes a base, may be positioned in association with the first delivery vehicle, i.e., the film/sponge combination. Upon administration at the desired body site, the acid and base may react to produce effervescence. In some other embodiments, a sponge material may be double-dipped in both an edible acid and a base and then incorporated into a delivery system. For instance, a portion of the sponge material could be dipped in an edible acid and the remaining portion dipped in a base. The acid and base will react to produce effervescence upon administration.

Suitable edible acids include, but are not limited to, citric acid, phosphoric acid, tartaric acid, malic acid, ascorbic acid and combinations thereof. Suitable bases include, but are not limited to, alkali metal carbonates, alkali metal bicarbonates, alkaline earth metal carbonates, alkaline earth metal bicarbonates and combinations thereof.

In some embodiments, the first and second delivery vehicles described above may be packaged together for consumer use. For instance, in some embodiments, a consumable product may include a container having one or more compartments. Any of the delivery systems described above may be housed within the compartments of the container. For instance, a mucoadhesive film having a cavity therein may be housed in one compartment. The cavity may be an open cavity, as described above. A second delivery vehicle, such as a tablet, may be housed in the same or a second compartment of the container. A consumer may open the package, remove the two delivery vehicles from the compartment(s), place the second delivery vehicle in the cavity of the mucoadhesive film and administer the delivery system by placing it against mucosal tissue at the desired body site. For example, the consumer may position the delivery system in the buccal cavity. Once the delivery system is positioned at the desired body site, the film may combine with moisture and adhere to the mucosal tissue. The active then may release from the second delivery vehicle and penetrate the mucosal tissue.

### Films

The films used in the delivery systems described herein may be produced by a combination of at least one polymer and a polar solvent, optionally including other fillers known in the art. The solvent may be water, a polar organic solvent including, but not limited to, ethanol, isopropanol, acetone, methylene chloride, or any combination thereof. The film may be prepared by utilizing a selected casting or deposition method and a controlled drying process. Such processes are described in more detail in commonly assigned U.S. application Ser. No. 10/074,272, filed on Feb. 14, 2002, and published as U.S. Patent Publication No. 2003/0107149 A1, the contents of which are incorporated herein by reference in their entirety. Alternatively, the films may be extruded as described in commonly assigned U.S. application Ser. No. 10/856,176, filed on May 28, 2004, and published as U.S. Patent Publication No. 2005/0037055 A1, the contents of which are incorporated herein by reference in their entirety.

The polymer included in the films may be water soluble, water swellable, water insoluble, or a combination of one or more either water soluble, water swellable or water insoluble polymers. The polymer may include cellulose or a cellulose derivative. Specific examples of useful water soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. In some embodiments, films formed from such water soluble polymers may be sufficiently water soluble to be dissolvable upon contact with bodily fluids.

Other polymers useful for incorporation into the films include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(a-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100L, believed to be 100% lactide having a melting point within the range of 338°-347° F. (170°-175° C.); lactide/glycolide 100L, believed to be 100% glycolide having a melting point within the range of 437°-455° F. (225°-23520 C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of active contained in the second delivery vehicle. Some actives may only require a few minutes for delivery through the mucosal tissue, whereas other actives may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water insoluble and/or biodegradable, as provided above. The inclusion of one or more polymers that are water-swellable, water insoluble and/or biodegradable may provide films with slower dissolution or disintegration rates than films formed from water-soluble polymers alone. As such, the film may adhere to the mucosal tissue for longer periods or time, such as up to several hours, which may be desirable for delivery of certain active components.

For instance, in some embodiments, the films may include polyethylene oxide alone or in combination with a second polymer component. The second polymer may be another water-soluble polymer, a water swellable polymer, a water insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropyl cellulose and/or hydroxypropylmethyl cellulose. In accordance with some embodiments, polyethylene oxide may range from about 20% to 100% by weight in the polymer component, more specifically about 30% to about 70% by weight, and even more specifically about 40% to about 60% by weight. In some embodiments, one or more water-swellable, water insoluble and/or biodegradable polymers also may be included in the polyethylene oxide-based film. Any of the water-swellable, water insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight.

The molecular weight of the polyethylene oxide also may be varied. In some embodiments, high molecular weight polyethylene oxide, such as about 4 million, may be desired to increase mucoadhesivity of the film. In some other embodiments, the molecular weight may range from about 100,000 to 900,000, more specifically from about 100,000 to 600,000, and even more specifically from about 100,000 to 300,000. In some embodiments, it may be desirable to combine high molecular weight (600,000 to 900,000) with low molecular weight (100,000 to 300,000) polyethylene oxide in the polymer component.

A variety of optional components and fillers also may be added to the films. These may include, without limitation: surfactants; plasticizers; polyalcohols; anti-foaming agents, such as silicone-containing compounds, which promote a smoother film surface by releasing oxygen from the film; thermo-setting gels such as pectin, carageenan, and gelatin, which help in maintaining the dispersion of components; inclusion compounds, such as cyclodextrins and caged molecules; coloring agents; and flavors. In some embodiments, an active component may be included in the film, in addition to the active component contained in the second delivery vehicle. Suitable active components for use in the film include any of those described below for use in the second delivery vehicle. The active contained in the film may be the same as or different from the active contained in the second delivery vehicle.

Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to 50% and more desirably within the range of 3% to 20% based on the weight of all film components.

Further additives may be inorganic fillers, such as the oxides of magnesium aluminum, silicon, titanium, etc. desirably in a concentration range of about 0.02% to about 3% by weight and desirably about 0.02% to about 1% based on the weight of all film components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂—, C₁₄—, C₁₆—, C₁₈—, C₂₀— and C₂₂— fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂—, C₁₄—, C₁₆—, C₁₈—, C₂₀— and C₂₂— fatty acids.

The total amounts used of the fats, mono-, di-glycerides and/or lecithins are up to about 5% and preferably within the range of about 0.5% to about 2% by weight of the total film composition.

It further may be useful to add silicon dioxide, calcium silicate, or titanium dioxide in a concentration of about 0.02% to about 1% by weight of the total composition. These compounds act as texturizing agents.

Lecithin is one surface active agent for use in the films described herein. Lecithin can be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other anti-foam and/or de-foaming agents may suitable be used.

As a related matter, simethicone and related agents may be employed for densification purposes. More specifically, such agents may facilitate the removal of voids, air, moisture, and similar undesired components, thereby providing denser, and thus more uniform films. Agents or components which perform this function can be referred to as densification or densifying agents. As described above, entrapped air or undesired components may lead to non-uniform films.

Simethicone is generally used in the medical field as a treatment for gas or colic in babies. Simethicone is a mixture of fully methylated linear siloxane polymers containing repeating units of polydimethylsiloxane which is stabilized with trimethylsiloxy end-blocking unites, and silicon dioxide. It usually contains 90.5-99% polymethylsiloxane and 4-7% silicon dioxide. The mixture is a gray, translucent, viscous fluid which is insoluble in water.

When dispersed in water, simethicone will spread across the surface, forming a thin film of low surface tension. In this way, simethicone reduces the surface tension of bubbles air located in the solution, such as foam bubbles, causing their collapse. The function of simethicone mimics the dual action of oil and alcohol in water. For example, in an oily solution any trapped air bubbles will ascend to the surface and dissipate more quickly and easily, because an oily liquid has a lighter density compared to a water solution. On the other hand, an alcohol/water mixture is known to lower water density as well as lower the water's surface tension. So, any air bubbles trapped inside this mixture solution will also be easily dissipated. Simethicone solution provides both of these advantages. It lowers the surface energy of any air bubbles that trapped inside the aqueous solution, as well as lowering the surface tension of the aqueous solution. As the result of this unique functionality, simethicone has an excellent anti-foaming property that can be used for physiological processes (anti-gas in stomach) as well as any for external processes that require the removal of air bubbles from a product.

In order to prevent the formation of air bubbles in the films, the mixing step can be performed under vacuum. However, as soon as the mixing step is completed, and the film solution is returned to the normal atmosphere condition, air will be re-introduced into or contacted with the mixture. In many cases, tiny air bubbles will be again trapped inside this polymeric viscous solution. The incorporation of simethicone into the film-forming composition either substantially reduces or eliminates the formation of air bubbles.

Simethicone may be added to the film-forming mixture as an anti-foaming agent in an amount from about 0.01 weight percent to about 5.0 weight percent, more desirably from about 0.05 weight percent to about 2.5 weight percent, and most desirably from about 0.1 weight percent to about 1.0 weight percent.

Any other optional components described in commonly assigned U.S. application Ser. Nos. 10/074,272 and 10/856,176, referred to above, also may be included in the films described herein.

### Active Components

As described above, the second delivery vehicle may include one or more active components. The active component contained in the second delivery vehicle may include, without limitation, pharmaceutical and cosmetic actives, drugs, medicaments, proteins, antigens or allergens such as ragweed pollen, spores, microorganisms, seeds, mouthwash components, flavors, fragrances, enzymes, preservatives, sweetening agents, colorants, spices, vitamins and combinations thereof. In some embodiments, the active component may be a substance that exhibits poor absorption or degradation when administered via the gastrointestinal route. Such actives include drugs, such as insulin, among others.

A wide variety of medicaments, bioactive active substances and pharmaceutical compositions may be included in the dosage forms of the present invention. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other preferred drugs for other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils, such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

An anti-oxidant may also be added to the film to prevent the degradation of an active, especially where the active is photosensitive.

Cosmetic active agents may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

Any of the actives set forth herein may be taste-masked prior to incorporation into the film, as set forth in International Application No. PCT/US02/32594, entitled “Uniform Films For Rapid Dissolve Dosage Form Incorporating Taste-Masking Compositions,” filed Oct. 11, 2002, and which published as WO 2003/030883 (claiming priority to U.S. Provisional Application No. 60/414,276 of the same title, filed Sep. 27, 2002), the contents both of which are incorporated by reference herein in their entirety.

1. A multi-vehicle delivery system comprising:
(a) a first delivery vehicle comprising at least one mucoadhesive film; and
(b) a second delivery vehicle comprising at least one active component,
wherein said second delivery vehicle is in association with said first delivery vehicle.

2. The delivery system of claim 1, wherein said mucoadhesive film at least partially surrounds said second delivery vehicle.

3. The delivery system of claim 1, wherein said second delivery vehicle is positioned within said mucoadhesive film.

4. The delivery system of claim 1, wherein said second delivery vehicle is adjacent to said mucoadhesive film.

5. The delivery system of claim 1, wherein said mucoadhesive film further comprises a cavity defined therein and said second delivery vehicle is positioned within said cavity.

6. The delivery system of claim 1, wherein said first delivery vehicle comprises a first mucoadhesive film and a second mucoadhesive film, and wherein said second delivery vehicle is positioned between said first and second mucoadhesive films.

7. The delivery system of claim 6, wherein said first mucoadhesive film is in at least partial face-to-face engagement with said second mucoadhesive film.

8. The delivery system of claim 7, wherein said first mucoadhesive film is fused to said second mucoadhesive film at said face-to-face engagement.

9. The delivery system of claim 1, wherein said mucoadhesive film comprises at least one polymer selected from the group consisting of water-soluble polymers, water swellable polymers, water insoluble polymers, biodegradable polymers and combinations thereof.

10. The delivery system of claim 1, wherein said mucoadhesive film comprises polyethylene oxide alone or in combination with a second polymer component.

11. The delivery system of claim 10, wherein said second polymer component is selected from the group consisting of water-soluble polymers, water swellable polymers, water insoluble polymers, biodegradable polymers and combinations thereof.

12. The delivery system of claim 1, wherein said mucoadhesive film is extruded.

13. The delivery system of claim 1, wherein said active component comprises a pharmaceutical active.

14. The delivery system of claim 1, wherein said active component comprises insulin.

15. The delivery system of claim 1, wherein said second delivery vehicle is selected from the group consisting of a tablet, capsule, powder, gel, liquid and combinations thereof.

16. The delivery system of claim 1, wherein said first delivery vehicle further comprises an edible acid and said second delivery vehicle further comprises a base.

17. The delivery system of claim 1, wherein said first delivery vehicle further comprises a sponge material in association with said mucoadhesive film.

18. The delivery system of claim 17, wherein said sponge material is affixed to said mucoadhesive film.

19. A multi-vehicle delivery system comprising:
(a) a first delivery vehicle comprising at least one mucoadhesive film; and
(b) a second delivery vehicle comprising at least one active component,
wherein said second delivery vehicle is adjacent to said first delivery vehicle.

20. The delivery system of claim 19, wherein said second delivery vehicle is adhered to said first delivery vehicle.

21. A multi-vehicle delivery system comprising:
(a) a first delivery vehicle comprising at least one mucoadhesive film, said first delivery vehicle having a cavity defined therein for accommodating a second delivery vehicle; and
(b) a second delivery vehicle positioned within said cavity, said second delivery vehicle comprising at least one active component.

22. The delivery system of claim 21, wherein said cavity comprises a closed cavity.

23. The delivery system of claim 21, wherein said cavity comprises an open cavity.

24. The delivery system of claim 23, further comprising a cover positioned over said open cavity.

25. A consumable product comprising:
(a) an outer container having one or more compartments; and
(b) a multi-vehicle delivery system housed in said one or more compartments, wherein said delivery system comprises:
(i) a first delivery vehicle comprising at least one mucoadhesive film, said first delivery vehicle having a cavity defined therein for accommodating a second delivery vehicle; and
(ii) a second delivery vehicle positioned within said cavity, said second delivery vehicle comprising at least one active component.

26. The consumable product of claim 25, wherein said outer container comprises a first compartment and a second compartment, and wherein said first delivery vehicle is housed in said first compartment and said second delivery vehicle is housed in said second compartment.

27. A method of making a multi-vehicle delivery system, comprising the steps of:
(a) providing a first delivery vehicle comprising a mucoadhesive film;
(b) forming a cavity in the mucoadhesive film; and
(c) positioning a second delivery vehicle within the cavity, wherein the second delivery vehicle comprises at least one active component.

28. The method of claim 27, wherein said step of providing a first delivery vehicle comprising extruding a mucoadhesive film.
